(19) United States
(12) Patent Application Publication
(10) Pub. No.: US 2018/0126146 A1
(43) Pub. Date: May 10, 2018

(54) CONNECTION DEVICE FOR A CATHETER, IN PARTICULAR A PERIPHERAL VENOUS CATHETER (II)

(71) Applicant: SICOS GMBH, Duesseldorf (DE)

(72) Inventors: André Reichardt, Apolda (DE); Christian Frauenrath, Juelich (DE); Kai Markus, Eschweiler (DE)

(21) Appl. No.: 15/309,492

(22) PCT Filed: Apr. 30, 2015
(86) PCT No.: PCT/DE2015/100178

§ 371 Date: Sep. 14, 2017

(30) Foreign Application Priority Data
May 8, 2014 (DE) 10 2014 106 454.2

(51) Int. Cl. A61M 39/16; A61M 5/165

(52) U.S. Cl. CPC A61M 39/162; A61M 2039/0072; A61M 2205/0205; A61M 5/165

(57) ABSTRACT

A connection device for a catheter, in particular a peripheral venous catheter, comprising: a) a main body having a main body infusion channel and a through-flow element having a sterilizing effect and forming at least one part of the main body infusion channel, wherein the through-flow element has an inlet opening, a liquid-tight circumferential wall, and an end wall which is arranged opposite the inlet opening and has at least one outlet opening, and b) an attachment body having an attachment body infusion channel and being detachably fixed on the main body, characterized in that the through-flow element enters a sealing press fit with the attachment body infusion channel through an attachment body fixed on the main body.

The invention relates to a connection device for a catheter, especially for a peripheral venous catheter, comprising a main body with a main body infusion channel and a sterilizing through-flow element forming at least a part of the main body infusion channel, wherein the through-flow element has an inlet opening, a liquid-tight circumferential wall and opposite to the inlet opening an end wall with at least one outlet opening, and an attachment body having an attachment body infusion channel and being detachably fixed on the main body.

A connection device for a peripheral venous catheter is known from EP 1 579 878 B1, which is particularly suitable for central venous catheters, which remain on the human body for long periods of time, in order to reduce the risk of septa caused by the entry of bacteria. Central venous catheters can be used e.g. for the artificial diet at short-bow syndrome. Such septa are associated with considerable personal disadvantages for the treated person. As a solution, the above-mentioned prior art proposes to provide as a sterilizing through-flow element a sieve element being made of silver, having numerous openings and being arranged in the infusion channel or, seen in the direction of the infusion flow, at the end of the infusion channel. The sieve element represents a barrier for bacteria and/or fungi and is also advantageous for the reason that the complete connection device together with the sieve element can be replaced in a very simple manner, in case the sieve element should become ineffective, e.g. as a result of clogging.

U.S. Pat. No. 5,782,808 discloses a connection device of the type initially mentioned. There, the main body comprising the through-flow element is integral. It is also disclosed to provide various surfaces of the main body as well as surfaces of the attachment body, which can be connected to the main body via a thread, with a sterilizing coating. In one embodiment, it is furthermore possible to provide also the inner surfaces of the through-flow element with the silver coating. For the sealing between main body and attachment body sealing rings are provided.

U.S. Pat. No. 5,049,139 discloses a connection device for a catheter in which a block of a water-soluble glass that is doped with silver, is arranged between two parts of the connection device, and has a passage for the infusion liquid. When used parts of the block dissolve in the infusion liquid so that the antibacterial properties of the silver can come into effect. It is also disclosed that the inserted glass block has an annular flange which is clamped between the two parts of the connection device. A sealing function at the location of the annular flange is not mentioned. Due to the solubility of the glass block in the infusion liquid a sealing function of this kind is not given, which is why other sealing measures are necessary. The sealing problem is not addressed.

It is an object of the present invention to provide a connection device of the type mentioned at the outset which is easy to produce and which can further improve the protective effect against sepses.

This object is achieved in a connection device of the type mentioned at the outset by the feature that the through-flow element undergoes a sealing press fit with the attachment body infusion channel when the attachment body is fixed to the main body.

Exemplary and advantageous embodiments of the connection device are defined in the dependent claims.

The through-flow element is impermeable in the circumferential wall for a liquid, in particular for an infusion fluid, which on these grounds can enter the connection infusion channel only through the at least one outlet opening of the end wall. With the sealing press fit between the through-flow element and the attachment body infusion channel, the tightness of the connection between the main body and the attachment body with respect to the infusion fluid can be ensured.

The main body and the attachment body can be fixed to each other e.g. via threads. In addition to the sealing press fit between the through-flow element and the attachment body infusion channel a sealing press fit between the main body and the attachment body can be provided.

With the sealing press fit between the through-flow element and the attachment body infusion channel, further sealing measures can be supported or become dispensable. With the press fit, production tolerances on the dimensions of the attachment body infusion channel can be absorbed to some extent.

In order to achieve the press fit, the through-flow element and the inner wall of the attachment body infusion channel have suitable shapes. For example, the inner wall of the attachment body infusion channel can taper in the direction of flow of the infusion liquid, for example, the inner wall can be formed conically with a certain opening angle at least in the area of the intended contact with the sterilizing through-flow element. In this case, the through-flow element, at least in the region of the expected contact with the inner wall of the attachment body infusion channel, may have a cylindrically shaped outer wall or as well taper in the direction of flow, wherein the diameter reduction of the outer side of the circumferential wall of the through-flow element per unit of length is less than the given per unit length diameter reduction of the inner wall of the attachment body infusion channel. In the case of a conical shape, the outer side of the circumferential wall of the through-flow element has a smaller opening angle than the inner wall of the attachment body infusion channel.

The choice of an elastic material for the through-flow element and/or for the attachment body infusion channel can be advantageous for the press fit.

The through-flow element can have a single outlet opening or two or more outlet openings in its end wall. A plurality of small outlet openings can also be provided, whereby the through-flow element has a sieve-like construction.

The through-flow element can be fixed in the main body. Preferably, the fixing of the flow element is detachably designed for easy interchangeability and/or for easier access for cleaning purposes.

However, it may also be advantageous to design the connection element according to the invention such that the main body comprising the through-flow element is integral. In this case, it can be advantageous if the main body comprises, in particular is made completely of a massive material which acts sterilizingly. A massive material is understood to be a material which has a shape stability without being applied to a carrier or a supporting substrate. Films or thin films applied to a substrate are thus not massive material. However, a massive material can be a matrix doped with foreign substances or impregnated with foreign elements, for example reinforcing elements, in particular fibers.

Manufacturing the integral main body from a sterilizing massive material can make a coating, in particular a coating with a sterilizing effect, redundant. The massive material can, for example, consist entirely of a single substance with a sterilizing component. Alternatively, the massive material may comprise a matrix of at least one material into which at least one sterilizing substance is incorporated, for example in the form of an additive or doping. The at least one matrix material, which can itself be without or with a sterilizing effect, can be, for example, a plastic. It is particularly advantageous if the entire main body consists of a uniform plastic material as a matrix with a sterilizing doping.

Sterilizing agents are in particular those agents and substances, which are in particular bactericidal, antibiotic and/or cytostatic. Applicable as a sterilizing agent or as a sterilizing substance are e.g. silver, copper or silver- and/or copper-containing alloys or silver- and/or copper-containing chemical compounds.

The connection device according to the invention can also be designed in such a way that at least one sterilizing insert body is arranged in the interior of the through-flow element. Such an insert body increases the sterilizing surface and can be used independently of the number of outlet openings. The at least one insert body can e.g. have a cruciform cross-section viewed in the direction of flow. The at least one insert body in the interior of the through-flow element can be separate and interchangeable or likewise integral with the through-flow element or part of the integral main body.

The at least one insert body can be made of the same material as the main body. However, the insert body or at least one of the insert bodies can also consist of a material other than that of the main body. As material of the at least one insert body, for example massive silver or copper, or a massive alloy containing silver and/or copper, or a sterilizing doped plastic may be suitable, wherein in case of several insert bodies these may consist of different materials.

The through-flow element and/or the sterilizing insert body can be, for example, of silver or coated on the surface with silver. Instead of silver, for example, a silver- or copper-containing alloy or a silver- or copper-containing chemical compound or other sterilizing agent can be used, for example a plastic with bactericidal, antibiotic and/or cytostatic additives. The additive may, for example, be silver or copper. Through-flow element and insert body can also consist of different materials.

The attachment body infusion channel may also have a sterilizing effect, for example by means of a suitable coating or sterilizing substances which have been incorporated into the wall delimiting the attachment body infusion channel, for example by doping. As a result, the sterilizing surface that is in direct contact with the liquid flowing through is enlarged, which can mean increased protection. In particular, the attachment body can comprise a sterilizing massive material, wherein all material variants of the massive material described in connection with the main body can also be advantageous for the attachment body. In addition, the attachment body can be designed in one piece.

The connection device according to the invention can also be designed in such a way that the attachment body has, at its end facing away from the main body, a coupling piece for connection to a cannula, in particular a butterfly cannula. In this way, the application of a catheter can be accelerated and simplified. Intermediate elements, for example, in the form of a hose, which may also be contaminated and the attachment of which is time consuming, become dispensable therewith.

However, it may also be advantageous to design the connection device according to the invention in such a way that the attachment body is part of a cannula, in particular a butterfly cannula.

In the following a preferred embodiment of the connection device according to the invention is presented on the basis of figures.

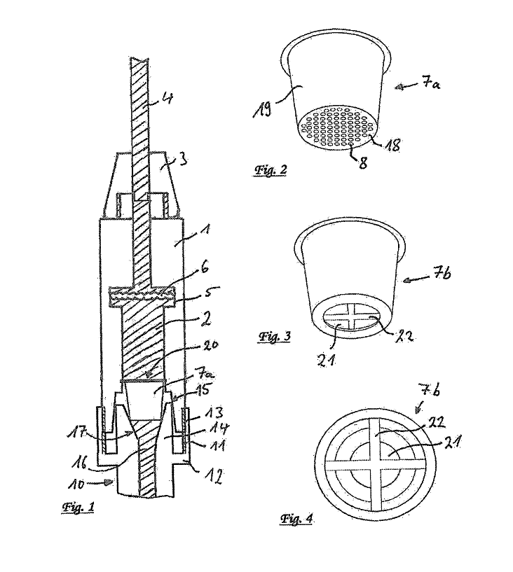

FIG. 1 shows a first embodiment of a connection device with a through-flow element fixed in the main body,

FIG. 2 shows a through-flow element with numerous outlet openings,

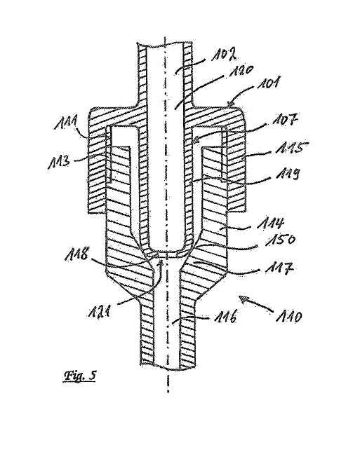

FIG. 3 shows a through-flow element with a sterilizing insert body in a perspective view,

FIG. 4 shows the through-flow element according to FIG. 3 in a top view and

FIG. 5 shows a second embodiment of a connection device with a one-piece main body.

FIG. 1 shows schematically a first connection device with a main body 1 through which a main body infusion channel 2 passes. The area penetrated by infusion fluid during operation is shown in FIG. 1 by hatching. The main body 1 is coupled to an infusion tube 4 via a connecting element 3. A widening 5 is provided in the main body infusion channel 2 and has a bacterial filter 6.

At the lower end of the main body infusion channel 2, a through-flow element 7a is fixed, which is shown in FIG. 2 enlarged in a perspective view obliquely from below. The through-flow element 7a has numerous outlet openings 8 solely at one end wall 18, while a circumferential wall 19 is of liquid-tight design. The end wall 18 can be—as shown—flat or have any other shape, e.g. also bulging. The through-flow element 7a consists of a sterilizing material and/or is coated with a sterilizing material. The sterilizing material may be, for example, silver or copper.

The sterilizing effect of the through-flow element 7a can be sufficient in order to be able to dispense with the illustrated bacterial filter 6.

By means of a nut element 12 having an inner thread 11 an attachment body 10 is fixed to an external thread 13 of the main body 1. The attachment body 10 has an engagement fitting 14 engaging in an end region 15 of the main body infusion channel 2. The engagement fitting 14 is made of a flexible material and dimensioned in such a way relative to the end region 15 of the main body 1 that, when fixing the attachment body 10, a press fit results between the engagement fitting 14 and the end region 15 of the main body 1.

With the attachment body 10 fixed, the through-flow element 7a projects into an attachment body infusion channel 16, which is expanded in an input region 17.

The circumferential wall 19 of the through-flow element 7a is conical, but has a significantly smaller opening angle than the inner wall of the conical engagement fitting 14. The connection between the main body 1 and the attachment body 10 results in a press fit between the through-flow element 7a and the engagement fitting 14. A sealing function is provided with the press fit. A sealing function in the connection between the attachment body 10 and the main body 1 is thus not required or is at least supported by the press fit between the through-flow element 7a and the attachment body infusion channel 16.

FIG. 3 shows a perspective view and FIG. 4 shows a top view of an alternative embodiment of a through-flow element 7b, which can be used instead of through-flow element 7a. Through-flow element 7b has only a single outlet opening 21, wherein a sterilizing cross-shaped insert body 22 is provided in the through-flow element 7b.

FIG. 5 shows a second connection device with a main body 101 having a main body infusion channel 102. In FIG. 5, unlike FIG. 1, the massive material of the bodies involved is shown hatched. The main body 101 is connected in a manner not shown here to an infusion hose, which is also not shown here. The main body 101 comprising a through-flow element 107 is integrally formed. The through-flow element 107 has an inlet opening 120 and a liquid-tight peripheral wall 119 and forms part of the infusion channel 102 of the main body 101.

An attachment body 110, having an attachment body infusion channel 116, can be fixed to the main body 101 via a coupling sleeve 115, having a thread 111 and surrounding a portion of the through-flow element 107. For this purpose, an engagement sleeve 114 belonging to the attachment body 110 and having a thread 13 is screwed into the coupling sleeve 115. When screwing the attachment body 110 in, an end piece 150 of the through-flow element 107 enters into a liquid-tight press fit with a conical inlet part 117 of the attachment body 110. The lower end wall 118 of the through-flow element 107 has an outlet opening 121 for the flow of the infusion liquid. Alternatively, the end wall 18 may also have a plurality of openings, in particular a plurality of sieve openings, as shown at the end wall 18 of the through-flow element 7a of FIG. 2.

The attachment body 110 can be connected to a discharging infusion tube (not shown here) or to a cannula (not shown here). The attachment body 110 can also be part of the cannula.

The through-flow element 107 can also have at least one insert body (not shown in FIG. 5) which can also have a sterilizing effect. The insert body could, for example, have a cruciform cross-section in the direction of flow, corresponding to the insert shown in FIGS. 3 and 4. Other shapes of the insert body are also possible. The insert body can be separate, in particular detachable. However, the insert body can also be integrally formed with the through-flow element 107 and thus with the residual main body 101.

The main body 101 with through-flow element 107 is made of a sterilizing massive material, for example comprising silver or copper or a plastic, the plastic having incorporated, for example in the form of inclusions or a doping, a sterilizing material, e.g. silver or copper.

### LIST OF REFERENCE NUMBERS

1 Main body
2 Main body infusion channel
3 Connecting element
4 Infusion tube
5 Widening
6 Bacterial filter
7 Through-flow element
8 Sieve opening
10 Attachment body
11 Inner thread
12 Nut element
13 External thread
14 Engagement fitting
15 End region
16 Attachment body infusion channel
17 Input region
18 End wall
19 Peripheral wall
20 Inlet opening
21 Outlet opening
22 Insert body 22
101 Main body
102 Main body infusion channel
107 Through-flow element
110 Attachment body
111 Thread
113 Thread
114 Engagement sleeve
115 Coupling sleeve
117 Inlet part
118 End wall
119 Peripheral wall
121 Outlet opening

1-12. (canceled)

13. A connector device for a catheter, especially for a peripheral venous catheter, comprising
a main body with a main body infusion channel and a sterilizing through-flow element forming at least a part of the main body infusion channel, wherein the through-flow element has an inlet opening, a liquid-tight circumferential wall and opposite to the inlet opening an end wall with at least one outlet opening, and
an attachment body having an attachment body infusion channel and being detachably fixed on the main body,
wherein, the through-flow element undergoes a sealing press fit with the attachment body infusion channel when the attachment body is fixed to the main body.

14. The connector device claimed in claim 13 wherein the attachment body infusion channel tapers in the direction of flow at least in an inlet part surrounding an end piece of the through-flow element.

15. The connector device claimed in claim 14 wherein the outer side of the circumferential wall of the through-flow element is cylindrical.

16. The connector device claimed in claim 14 wherein the outer side of the circumferential wall of the through-flow element is conical with a diameter decreasing towards the end wall.

17. The connector device claimed in claim 13 wherein at least one sterilizing insert body is arranged inside the through-flow element.

18. The connector device claimed in claim 13 wherein the attachment body has a sterilizing effect, at least on a section of the attachment body infusion channel.

19. The connector device claimed in claim 13 wherein the main body is integral.

20. The connector device claimed in claim 13 wherein the through-flow element consists of a massive material with sterilizing effect.

21. The connector device claimed in claim 13 wherein the entire main body consists of a massive material with a sterilizing effect.

22. The connector device claimed in claim 20 wherein the massive material comprises a matrix of a plastic material, the matrix being doped with a sterilizing substance.

23. The connector device claimed in claim 21 wherein the massive material comprises a matrix of a plastic material, the matrix being doped with a sterilizing substance.

24. The connector device claimed in claim 13 wherein the attachment body has, at its end facing away from the main body, a coupling piece for connection to a cannula, in particular a butterfly cannula.

25. The connector device claimed in claim 13 wherein the attachment body is part of a cannula, in particular a wing cannula.
